(19) United States
(12) Patent Application Publication
(10) Pub. No.: US 2006/0099632 A1
(43) Pub. Date: May 11, 2006

(54) Methods for preparation of a nucleic acid for analysis

(75) Inventor: Joseph A. Sorge, Wilson, NY (US)

Correspondence Address: PALMER & DODGE, LLP;KATHLEEN M. WILLIAMS / STR, 111 HUNTINGTON AVENUE, BOSTON, MA, 02199, US

(73) Assignee: Stratagene California

(21) Appl. No.: 11/300,139

(22) Filed: Dec. 14, 2005

Related U.S. Application Data
(63) Continuation of application No. 10/910,957, filed on Aug. 4, 2004, now abandoned.
Application No. 10/910,957 is a continuation of application No. 10/034,870, filed on Nov. 1, 2001, now abandoned.
(60) Provisional application No. 60/247,335, filed on Nov. 10, 2000.

(51) Int. Cl. C12Q 1/68; C12P 19/34

(52) U.S. Cl. 435/6; 435/91.2

(57) ABSTRACT

The present invention provides a method of preparing a nucleic acid sample comprising template nucleic acid and synthetic nucleic acid for analysis wherein prior to analysis the nucleic acid sample is treated with a substance which selectively cleaves the template nucleic acid without substantially cleaving the synthetic nucleic acid. The invention further provides a method for improving the analysis of capillary-based DNA sequencing reactions, amplification reactions, and/or transcription reactions, wherein after the reaction, the nucleic acid sample comprising template nucleic acid and synthetic nucleic acid is treated with a substance which selectively cleaves the template without substantially cleaving the synthetic nucleic acid.

### RELATED APPLICATIONS

This application is a Continuation of application Ser. No. 10/910,957, filed on Aug. 4, 2004, which claims priorty to appliation Ser. No. 10/034,870, filed on Nov. 1, 2001, which claims the benefit of U.S. Provisional Application Serial No. 60/247,335, filed Nov. 10, 2000. Theses applications are incorporated herein by reference in their entirety.

### BACKGROUND OF THE INVENTION

Analysis of nucleic acid molecules is a principle technique in the advancement of molecular biology, genetic discovery, and the development of new and improved therapeutics. Nucleic acids may be analyzed by a variety of means, generally comprising the separation of nucleic acid molecules based on size against an electrolytic matrix. Most commonly, the matrix is comprised of a cross-linked, or non-cross-linked polymer through which the nucleic acids travel; small nucleic acid molecules move rapidly through the matrix, while larger molecules move more slowly through the matrix. One problem encountered in the separation of nucleic acid is that the presence of very large nucleic acid molecules can clog the matrix and block the migration of other nucleic acids. This results in a decrease in the quantity of data which may be obtained from a given analysis, and a reduction in the quality of the data.

For example, an emerging technique in the field of DNA sequencing is the use of capillary electrophoresis as the basis for separation of DNA molecules following a sequencing reaction. One of the problems with capillary sequencers is that they are very sensitive to the amount of DNA loaded into the capillary. Too much DNA, or DNA molecules that are too large can clog the capillary yielding unusable sequencing data. As is often the case, the sequencing reaction utilizes double stranded plasmid DNA (approximately 3 kb) as a template for cycle sequencing, thus the plasmid DNA is loaded into the capillary along with the synthetic product of the sequencing reaction. The larger vector DNA can increase the viscosity of the sample within the capillary and effectively clog the capillary.

Therefore, there exists a need in the art for a method of reducing the size and thus viscosity of a nucleic acid sample prior to analysis by methods such as capillary electrophoresis. The method of reducing the size must be selective for the plasmid template nucleic acid, as any manipulation of the size or molecular weight of the synthetic nucleic acid may yield inaccurate, or erroneous analysis.

### SUMMARY OF THE INVENTION

The present invention provides a method of preparing a nucleic acid sample for an analytical procedure, the sample comprising template nucleic acid and synthetic nucleic acid, wherein the template and synthetic nucleic acid comprise DNA, comprising treating the sample with a substance that cleaves the template nucleic acid without substantially cleaving the synthetic nucleic acid.

In a preferred embodiment, the template nucleic acid and synthetic nucleic acid consist essentially of DNA.

In a further preferred embodiment, the template nucleic acid and synthetic nucleic acid consist of DNA.

In one embodiment, the invention further comprises subjecting the treated sample to the analytical procedure.

In a further embodiment, the analytical procedure is selected from the group comprising gel electrophoresis, anion-exchange chromatography, size-exclusion chromatography, pulse-field electrophoresis, polyacrylamide gel electrophoresis, sieving gel electrophoresis, capillary electrophoresis, Northern analysis, Southern analysis, or DNA sequencing.

The present invention further comprises a capillary-based DNA sequencing reaction wherein a nucleic acid sample is generated comprising template nucleic acid and synthetic nucleic acid, the improvement whereby after the sequencing reaction and prior to electrophoretic analysis of the nucleic acid sample, the sample is treated with a substance that cleaves the template nucleic acid and does not substantially cleave the synthetic nucleic acid.

The present invention further comprises an amplification reaction wherein a nucleic acid sample is generated comprising template nucleic acid and synthetic nucleic acid, the improvement whereby after the amplification reaction and prior to analysis of the nucleic acid sample, said sample is treated with a substance that cleaves the template nucleic acid and does not substantially cleave the synthetic nucleic acid.

The present invention further comprises a transcription reaction wherein a nucleic acid sample is generated comprising template nucleic acid and synthetic RNA, the improvement whereby after the transcription reaction and immediately prior to the analysis of the RNA sample, said nucleic acid sample is treated with a substance that cleaves the template nucleic acid and does not substantially cleave the RNA.

In preferred embodiments, the synthetic nucleic acid is synthesized from said template.

In further embodiments, the synthesized nucleic acid is synthesized in a reaction selected from the group comprising sequencing reactions, self-sustained sequence replication amplification, transcription based amplification, strand displacement amplification, ligation chain reaction, nucleic acid-based amplification, or oligonucleotide ligation assay.

In a further preferred embodiment, the synthesized nucleic acid is synthesized in a sequencing reaction.

In a further embodiment, the substance is a restriction enzyme.

In a preferred embodiment, the restriction enzyme specifically cleaves nucleic acid comprising modified residues, without substantially cleaving unmodified residues.

In a further embodiment, the restriction enzyme specifically cleaves nucleic acid comprising unmodified residues, without substantially cleaving modified residues.

In a still further embodiment, the restriction enzyme specifically cleaves double stranded nucleic acid, without substantially cleaving single stranded nucleic acid.

In another embodiment, the template nucleic acid is a double stranded nucleic acid.

In another embodiment, the synthetic nucleic acid is a single stranded nucleic acid.

In a further embodiment, the double-stranded template is produced in cells which incorporate methylated adenine residues into DNA molecules during replication.

In a still further preferred embodiment, the cell is a dam+E.coli cell.

As used herein, “analytical procedure” refers to any process by which at least a portion of the synthetic product of a nucleic acid template is subjected to a technique which permits determination of one or more of its molecular mass, molecular weight, molecular size, purity, length, molecular sequence, and/or concentration. An “analytical procedure” may refer to the separation of nucleic acids found in the sample, whether the nucleic acids separated are the template and synthetic nucleic acids, or whether the nucleic acids to be separated comprise the synthetic nucleic acids only. Such nucleic acids may be DNA and RNA, different sizes of DNAs and/or RNAs, or single nucleotides and polynucleotides. The separation of nucleic acids may be accomplished by passing electrical current across a matrix into which the nucleic acid is placed, i.e., gel electrophoresis, or by other chromatographic or physical means, including, but not limited to anion-exchange chromatography, size-exclusion chromatography, agarose gel electrophoresis, pulse-field electrophoresis, polyacrylamide gel electrophoresis, sieving gel electrophoresis, or capillary electrophoresis. However, separation of nucleic acids is step in a number of molecular biological techniques including, but not limited to Northern analysis, Southern analysis, DNA sequencing, etc. Thus “analytical procedure”, in addition to referring to the actual separation of nucleic acids, also refers to any molecular biological technique or series of techniques which incorporates such separation. In preferred embodiments, an “analytical procedure” is a method of evaluating a synthetic nucleic acid product in which the template from which the synthetic product is derived may interfere with the evaluation of synthetic product.

As used herein, “improved”, as it refers to an analytical procedure, refers to any increase in the quantity or quality of data which is obtained from subjecting a nucleic acid sample to a technique which permits determination of one or more of its molecular mass, molecular weight, molecular size, purity, length, molecular sequence, and/or concentration of synthetic nucleic acids following a given analytical procedure. For example, if the analytical procedure is capillary based DNA sequencing, an “improvement” in the sequencing could be the resolution of a higher number of bases from a given sample. In preferred embodiments, “improved” capillary based DNA sequencing would resolve about 10-20% more bases than non-“improved” capillary based sequencing, preferably about 20-50%, more preferably about 50-80%, and most preferably about 80-100% more bases. Similarly in non-capillary sequencing, such as polyacrylamide gel slab sequence analysis, “improved” sequencing would resolve about 10-20% more bases than non-improved sequencing, preferably about 20-50%, more preferably about 50-80%, and most preferably about 80-100% more bases.

Alternatively, for analytical procedures wherein a nucleic acid sample is assessed by gel electrophoresis, an improvement in the analytical procedure refers to an increase in the signal intensity and/or sample resolution. For example, a nucleic acid sample is analyzed by gel electrophoresis wherein nucleic acid molecules of a given size migrate a certain distance through the gel, so as to create a band of similarly sized nucleic acid molecules. The gel may be stained with a dye such as ethidium bromide which intercalates into the nucleic acid and fluoresces under UV illumination. The nucleic acid bands may thus be photographed, and the photographs scanned into a computer where the intensity of pixels for each band is determined using analysis software such as NIH Image (National Institutes of Health, Bethesda, Md.) or Scion Image (Scion Corp., Frederick, Md.). A plot may be generated of pixel intensity (y-axis) versus area (x-axis; wherein the nucleic acid band is circumscribed by the user and the area of the circumscribed band is calculated), and the area under the resulting bell-shaped curve may be calculated. An “improvement” in sample resolution is defined as a condition under which the area under the curve is decreased without reducing the amplitude of the curve. Preferably the area under the curve is reduced by 10-20%, more preferably 20-30%, and still more preferably 30-50%. An “improvement” in signal intensity is defined as an increase in the amplitude of the curve along the y-axis. Preferably, the amplitude of the curve is increased by 10-20%, more preferably 20-30%, and still more preferably 30-50%.

As used herein “cleavage” and/or “cleaves” refers to the breakage of the phosphodiester linkage between nucleotide residues in a polynucleotide chain. As used herein “cleavage” or “cleaves” refers to the breakage of at least one phosphodiester bond in a polynucleotide chain, i.e., a single bond, or multiple bonds in a chain. As used herein “cleavage” and/or “cleaves” refers to a reduction in the molecular weight of the nucleic acid which is being “cleaved”, by at least 10%, more preferably between 10-30%, more preferably between 30-70%, and still more preferably between 70-90%, as measured by molecular weight in agarose gel electrophoresis using molecular weight standards to determine changes in molecular weight. For example, if a given plasmid DNA molecule is cleaved twice, to yield two fragments of the same length, then the molecular weight of each fragment has been reduced by approximately 50% from the molecular weight of the original plasmid. In the context of the present invention which pertains to the cleavage of a large (2-50 kb) nucleic acid template, whether that be a linear DNA or RNA or a circular DNA or RNA molecule, such as plasmid DNA, “cleavage” refers to the breakage of a single bond in the plasmid, resulting in the linearization of the plasmid, or to the breakage of multiple bonds in the plasmid, resulting in a number of linear fragments of the plasmid. As used herein, “cleavage” further refers to the breakage of one or more phosphodiester bonds in at least 10% of the nucleic acid intended to be cleaved, preferably 10-30%, more preferably 30-70% and still more preferably 70-100%.

“Cleavage” may refer to the breakage of a single phosphodiester bond in a circular plasmid, resulting in a linear, double stranded nucleic acid. Under such conditions, the apparent molecular weight of the nucleic acid is said to be reduced as evidenced by an increase in migration in gel electrophoresis. Typically, when nucleic acid samples, which contain large circular plasmid nucleic acid, are subjected to gel electrophoresis, a large amount (30-80%) of the plasmid may remain in the loading well and not migrate into the gel. Accordingly, “cleavage” of the plasmid would result in migration of the “cleaved” plasmid into the gel and a reduction in the amount of nucleic acid which is retained in the loading well as can be determined by techniques known to those of skill in the art. Following cleavage, the amount of plasmid retained in the loading well is preferably reduced by at least 10%, more preferably 10-30%, more preferably 30-60%, more preferably 60-80%, and still more preferably 80-100%.

Alternatively, a double stranded nucleic acid template can be said to have been “cleaved” if there is improvement in the subsequent analysis of the nucleic acid sample, according to the methods of the invention

As used herein, “without substantially cleaving” refers to the breakage of not more than between 3-5 phosphodiester bonds in a nucleic acid chain, preferably not more than between 2-3, and most preferably one or none. Alternatively, “without substantially cleaving” may refer to cleavage of not more than 10% of a nucleic acid with respect to the total amount of nucleic acid present in the synthetic reaction.

As used herein, “immediately prior to the analysis” means that there are no intervening nucleic acid digestion reactions, particularly with either DNase or RNase, between the synthetic reactions of the present invention and treatment with the substances which cleave the template nucleic acid as described herein.

As used herein, “plasmid” refers to a circular, double stranded, extrachromasomal genetic element composed of DNA or RNA, or cDNA, or modified DNA found in both prokaryotic and eukaryotic cells. “Plasmids” of the invention can also be supercoiled. “Plasmids”, useful in the present invention, can be derived from numerous host organisms known to those of skill in the art including, but not limited to lambda bacteriophage, M13 bacteriophage, E. coli, S. cerevisiae, or a synthetic plasmid which can be replicated in a prokaryotic and/or eukaryotic host cell. The size of a “plasmid” useful in the present invention can vary depending on the source from which the plasmid is derived and the size of nucleic acid construct, useful in the invention, which is inserted into the plasmids. Plasmids of the invention can range in size from about 2 kb to 50 kb.

As used herein, “template” refers to a polynucleotide chain of either DNA or RNA, which may be single or double stranded, that may be utilized during DNA replication, transcription, and/or another synthetic process as a guide to the synthesis of a second polynucleotide chain with a complementary base sequence. In preferred embodiments of the present invention a “template” nucleic acid is double stranded. A “template” nucleic acid may be genomic DNA, or total RNA, mRNA, a plasmid, or yeast artificial chromosome, or may be any nucleic acid which possesses characteristics such that it may be replicated, transcribed, or amplified in vitro.

As used herein, “synthetic nucleic acid” refers to a nucleic acid with a complementary nucleotide sequence to the plasmid template from which it was generated. A “synthetic nucleic acid” as used herein, may be generated by any synthetic reaction known in the art.

As used herein, “synthetic process” or “synthetic reaction” is any process, known to those of skill in the art, by which a template nucleic acid is utilized as a guide in the generation of a second nucleic acid with a complementary nucleotide sequence to the template from which it was generated. A “synthetic reaction” useful in the present invention may be selected from the group comprising sequencing reactions, self-sustained sequence replication amplification, transcription based amplification, strand displacement amplification, ligation chain reaction, polymerase chain reaction, oligonucleotide ligation assay, or nucleic acid-based amplification.

As used herein, “modified residues” refers to any postsynthetic addition, either occurring naturally within the cell, or induced in vitro by one of skill in the art, such as following an amplification reaction, in prokaryotic and/or eukaryotic cells, of small chemical moieties to an intact DNA or RNA polymer. In preferred embodiments of the present invention, “modified residues” are residues to which a methyl group (—CH₃) has been added, preferably at position C5 of cytosine or position N6 of adenosine, however bases with methyl groups at additional positions or on bases other than cytosine and adenosine, including, but not limited to 2′-O-methylcytidine, 2′-O-methylguanosine, 1-methyladenosine, 1-methylguanosine, 2,2-dimethylguanosine, 2-methyladenosine, 2-methylguanosine, 3-methylcytidine, 5-methylcytidine, and 7-methylguanosine are also considered “modified residues” according to the invention. Additionally, in certain embodiments, “modified residues” include any purine or pyrimidine ring except the usual A, T, G, or C including, but not limited to inosine, queuosine, wyosine, beta, D-mannosylqueuosine, 2-methylthio-N6-isopentenyladenosine, wybutoxosine, 2-thiocytidine, and wybutosine.

The invention provides a method for improving the analysis of nucleic acids following synthetic or amplification reactions by selectively cleaving the template nucleic acid within a sample, allowing for an increase in the quality and/or quantity of data which may be obtained about the nucleic acid sample.

### DETAILED DESCRIPTION

The present invention teaches a method of preparing a nucleic acid sample for an analytical procedure wherein the sample comprises a template nucleic acid and a synthetic nucleic acid, and wherein the sample is treated with a substance that cleaves the template nucleic acid without significantly cleaving the synthetic nucleic acid.

Synthetic Reactions

Synthetic reactions of the present invention relate to both the generation of plasmid template nucleic acid, and the generation of synthetic products from the plasmid template.

Synthesis of Template

Template nucleic acids of the present invention include, but are not limited to plasmids, cosmids, episomes, genomic DNA, genomic DNA fragments, cloned DNA fragments, amplification products, cloned DNA, amplification products, PCR products and/or reverse transcription products. Template nucleic acids useful in the present invention may be obtained from a biological sample (e.g., tissues, fluids, cells) or may be synthesized. Preparation of template nucleic acid is taught in a number of texts and/or laboratory manuals including Molecular Cloning (Maniatis et. al. (1982), Cold Spring Harbor) or Short Protocols in Molecular Biology (Ausubel et. al. (1995) 3^{rd} Ed. John Wiley & Sons, Inc.).

In one embodiment of the invention, the template nucleic acid is plasmid DNA. Plasmid template nucleic acid molecules of the present invention may be produced by any method known to those of skill in the art. Methods for the production of plasmid template nucleic acid are available through a number of texts and laboratory manuals including Short Protocols in Molecular Biology (Ausubel et. al. (1995) 3^{rd} Ed. John Wiley & Sons, Inc.). Briefly, a nucleic acid, gene, gene fragment, etc. may be cloned into an acceptable plasmid which is then introduced into a host organism such as E. coli, where the plasmid is replicated. The plasmid may then be purified from the bacterial cells and used for synthesis of synthetic nucleic acids in amplification reactions as described herein. Plasmids for cloning of nucleic acids are numerous, and include but are not limited to pBR322, pGEM-3Z, [symbol] X174, pGEM-4Z, pSP72, pSP73, pGEMEX, M13, BluescriptII, pBC, pSVK3, pBS, pcDNAII, pEMBL18/19, pfdA/B, pIB124, pICEM, pSELECT, pAM18/19, pAT153, pUCBM20/21, and SP64. The plasmid may be selected based on the particular nucleic acid to be cloned, the host organism into which the plasmid is to be cloned, or specific properties of the plasmid such as antibiotic resistance. The plasmid is cleaved with one or more restriction enzymes to linearize the plasmid. The nucleic acid to be cloned is also cleaved with either the same restriction enzymes, or different enzymes that will, nonetheless, produce a nucleic acid which may be ligated into the plasmid. The nucleic acid is then ligated to the plasmid using DNA ligase under appropriate conditions, such that the nucleic acid is incorporated into the, now circular, recombinant plasmid. Host cells, including but not limited to E. coli and various strains thereof, are then transformed with the recombinant plasmid by any technique known in the art, including, but not limited to heat shock, electroporation, lippofection, or calcium-phosphate precipitation. The transformed host cells are then grown in appropriate medium, such as Luria Broth, for between 12 and 24 hours at approximately 37° C.

The host cells are subsequently removed from the growth medium by centrifugation, and lysed under alkaline conditions to release their cellular contents. The nucleic acid is subsequently precipitated out of solution with ethanol, and purified by CsCl centrifugation. The purified plasmid may then be used in any of the synthetic or amplification reactions described herein.

In one aspect of the invention, the template nucleic acid is plasmid DNA, synthesized in dam⁺ E. coli which selectively methylate adenine residues, and the nucleic acid is cleaved by a restriction enzyme which selectively cleaves methylated nucleic acid. The selective cleavage of the template nucleic acid thus provides an improvement in the analysis of the synthetic product synthesized from the template.

E. coli are bacterial cells widely used in the laboratory to clone myriad genes, or nucleic acid fragments. Most E. coli employed in the laboratory contain three site-specific DNA methylases, including the methylase encoded by the dam gene (Dam methylase). The Dam methylase transfers a methyl group from S-adenosylmethonine to the N⁶ position of the adenine residues in the sequence GATC (Marinus and Morris (1973) J Bacteriol. 114:1143; Geier and Modrich (1979) J. Biol. Chem. 254: 1408). Bacterial methylases can be useful biological tools, as they can provide specific nucleic acid variations which are useful in laboratory manipulation of DNA. For example, some or all of the sites for a restriction endonuclease may be resistant to cleavage when isolated from E. coli strains expressing the Dam methylase. This results from the inability of certain restriction enzymes to cleave DNA when one or more nucleic acid residues in its recognition site are methylated. This occurs when the recognition sites for methylation and endonuclease cleavage for a given enzyme overlap. In addition to restriction endonucleases which exhibit decreased site recognition following methylation, there are other endonucleases, such as Dpn I which selectively cleave nucleic acid only when their recognition site is methylated.

E. coli strains therefore, with an active Dam methylase are useful in the present invention as the nucleic acid synthesized from such cells will be methylated at the appropriate residues, and thus can be distinguished from homologous or complementary nucleic acid which is not derived from dam⁺ E. coli.

Synthetic Reactions

In an embodiment of the present invention, a nucleic acid sample of the invention comprises both template nucleic acid and synthetic nucleic acid, wherein there is selective cleavage of the template nucleic acid without significant cleavage of the synthetic nucleic acid.

Accordingly, template nucleic acid may be derived from any source known in the art including, but not limited to plasmids, cosmids, episomes, genomic DNA, genomic DNA fragments, cloned DNA fragments, amplification products, cloned DNA, amplification products, PCR products, reverse transcription products, or in preferred embodiments of the invention, from dam⁺ E. coli, is utilized in the generation of a synthetic nucleic acid by processes including, but not limited to sequencing reactions, transcription based amplification, strand displacement amplification, ligation chain reaction, or nucleic acid-based amplification.

Polymerase Chain Reaction

In preferred embodiments, the synthetic nucleic acid, useful in the invention is synthesized by polymerase chain reaction (PCR). The PCR technique is widely known and understood by those of skill in the art to be useful in the production of a large quantity of synthetic nucleic acid from a limited amount of single- or double-stranded nucleic acid (see U.S. Pat. No. 4,683,195, herein incorporated by reference).

The specific synthetic nucleic acid sequence is produced by using the nucleic acid containing that sequence (plasmid template) as a template. If the template nucleic acid contains two strands, it is necessary to separate the strands of the nucleic acid before it can be used as the template, either as a separate step or simultaneously with the synthesis of the primer extension products. This strand separation can be accomplished by any suitable denaturing method including physical, chemical or enzymatic means. One physical method of separating the strands of the template nucleic acid involves heating the nucleic acid until it is completely (>99%) denatured. Typical heat denaturation may involve temperature ranging from about 80° C. to 105° C. for times ranging from about 1 to 10 minutes. Strand separation may also be induced by an enzyme from the class of enzymes known as helicases or the enzyme RecA, which has helicase activity and in the presence of riboATP is known to denature DNA. The reaction conditions suitable for separating the strands of nucleic acids with helicases are described by Cold Spring Harbor Symposia on Quantitative Biology, Vol. XLIII “DNA: Replication and Recombination” (New York: Cold Spring Harbor Laboratory, 1978), B. Kuhn et al., “DNA Helicases”, pp. 63-67, and techniques for using RecA are reviewed in C. Radding, Ann. Rev. Genetics, 16:405-37 (1982).

If the template nucleic acid is single stranded, its complement is synthesized by adding one or two oligonucleotide primers thereto. If an appropriate single primer is added, a primer extension product is synthesized in the presence of the primer, an agent for polymerization and the four nucleotides described below. The product will be partially complementary to the single-stranded nucleic acid and will hybridize with the nucleic acid strand to form a duplex of unequal length strands that may then be separated into single strands as described above to produce two single separated complementary strands. Alternatively, two appropriate primers may be added to the single-stranded nucleic acid and the reaction carried out.

If the original nucleic acid (template) constitutes the sequence to be amplified, the primer extension product(s) produced will be completely complementary to the strands of the original nucleic acid and will hybridize therewith to form a duplex of equal length strands to be separated into single-stranded molecules.

When the complementary strands of the nucleic acid or acids are separated, whether the nucleic acid was originally double or single stranded, the strands are ready to be used as a template for the synthesis of additional nucleic acid strands. This synthesis can be performed using any suitable method. Generally it occurs in a buffered aqueous solution, preferably at a pH of 7-9, most preferably about 8. Preferably, a molar excess (for cloned nucleic acid, usually about 1000:1 primer:template, and for genomic nucleic acid, usually about 10⁶:1 primer:template) of the two oligonucleotide primers is added to the buffer containing the separated template strands. The amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleoside triphosphates dATP, dCTP, dGTP and TTP are also added to the synthesis mixture in adequate amounts and the resulting solution is heated to about 90°-100° C. for from about 1 to 10 minutes, preferably from 1 to 4 minutes. After this heating period the solution is allowed to cool to from 20°-40° C., which is preferable for the primer hybridization. To the cooled mixture is added an agent for polymerization, and the reaction is allowed to occur under conditions known in the art. This synthesis reaction may occur at from room temperature up to a temperature above which the agent for polymerization no longer functions efficiently. Thus, for example, if DNA polymerase is used as the agent for polymerization, the temperature is generally no greater than about 45° C. Preferably an amount of dimethylsulfoxide (DMSO) is present which is effective in detection of the signal or the temperature is 35°-40° C. Most preferably, 5-10% by volume DMSO is present and the temperature is 35°-40° C. For certain applications, where the sequences to be amplified are over 110 base pair fragments, such as the HLA DQ- [symbol] or - [symbol] genes, an effective amount (e.g., 10% by volume) of DMSO is added to the amplification mixture, and the reaction is carried at 35°-40° C., to obtain detectable results or to enable cloning.

The agent for polymerization may be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, reverse transcriptase, and other enzymes, including heat-stable enzymes, which will facilitate combination of the nucleotides in the proper manner to form the primer extension products which are complementary to each nucleic acid strand. Generally, the synthesis will be initiated at the 3′ end of each primer and proceed in the 5′ direction along the template strand, until synthesis terminates, producing molecules of different lengths. There may be agents, however, which initiate synthesis at the 5′ end and proceed in the other direction, using the same process as described above.

The newly synthesized strand and its complementary nucleic acid strand form a double-stranded molecule which is used in the succeeding steps of the process. In the next step, the strands of the double-stranded molecule are separated using any of the procedures described above to provide single-stranded molecules.

New nucleic acid is synthesized on the single-stranded molecules. Additional polymerase, nucleotides and primers may be added if necessary for the reaction to proceed under the conditions prescribed above. Again, the synthesis will be initiated at one end of the oligonucleotide primers and will proceed along the single strands of the template to produce additional nucleic acid. After this step, half of the extension product will consist of the specific nucleic acid sequence bounded by the two primers.

The steps of strand separation and extension product synthesis can be repeated as often as needed to produce the desired quantity of the specific nucleic acid sequence. As will be described in further detail below, the amount of the specific nucleic acid sequence produced will accumulate in an exponential fashion.

Sequencing Reactions

In a further embodiment of the invention, the synthetic nucleic acid may be synthesized by a sequencing reaction analogous to the “Sanger” or “dideoxy” DNA sequencing method (Sanger et. al., (1977) Proc. Natl. Acad. Sci. USA 74: 5463, which is incorporated herein by reference). This method relies upon the template-directed incorporation of nucleotides onto an annealed primer by a DNA polymerase from a mixture containing deoxy- and dideoxynucleotides. The incorporation of dideoxynucleotides results in chain termination, the inability of the enzyme to catalyze further extension of that strand. Subsequent electrophoretic separation of reaction products results in a “ladder” of extension products wherein each extension product ends in a particular dideoxynucleotide complementary to the nucleotide opposite it in the template. Extension products may be detected in several ways, including for example, the inclusion of isotopically-or fluorescently-labeled primers, deoxynucleotide triphosphates or dideoxynucleotide triphosphates in the reaction.

In preferred embodiments, nucleic acids of the present invention are synthesized by thermal cycle dideoxy (Sanger) sequencing reactions. Thermal cycle sequencing is a method by which a dideoxy sequencing reaction mixture is subjected to repeated rounds of denaturation, annealing, and synthesis steps, similar to PCR, resulting in linear amplification of the sequencing products. The plasmid template nucleic acid may be double or single stranded prior to the initiation of sequencing, and thus double stranded template may be converted to single stranded nucleic acid by alkali denaturation, or extreme heat as described above in the section on PCR.

The thermal sequencing may be carried out using protocols known to those of skill in the art and available in numerous texts, and laboratory manuals such as Short Protocols in Molecular Biology (Ausubel et. al. (1995) 3^{rd} Ed. John Wiley & Sons, Inc.). Briefly, four separate reactions are initiated, each specific for one of the four deoxyribonucleotides. To each reaction is added nucleic acid specific primers, thermostable DNA polymerase, such as Taq polymerase, [ [symbol] -³²P, [symbol] -³⁵S, or [symbol] -³³P]dATP, dNTPs, ddNTPs each added specifically to its corresponding reaction, and sequencing buffer. The reaction mix is then amplified for 20 cycles under the following thermal cycle conditions: 95° C., 20 s; 55° C/, 20 s; 720 C, 20 s. The samples are then analyzed by agarose gel electrophoresis, or capillary gel electrophoresis as described below. It should be noted that Sanger sequencing may be carried out by alternate protocols to the one described above, or using commercially available sequencing kits (i.e., Life Technologies, Rockville, Md.) known to those of skill in the art, all of which may be used with the present invention.

Transciption Based Amplification

In another embodiment of the invention, a synthetic nucleic acid can be synthesized from a plasmid nucleic acid using the method of transcription based amplification (TAS). The TAS system involves the use of primers that encode a promoter to generate DNA copies of a target strand and the production of RNA copies from the DNA copies using an RNA polymerase (U.S. Pat. No. 4,683,202, incorporated herein by reference).

The synthetic nucleic acid is produced by using the nucleic acid containing that sequence (plasmid template) as a template. If the template nucleic acid contains two strands, it is necessary to separate the strands of the nucleic acid before it can be used as the template, either as a separate step or simultaneously with the synthesis of the primer extension products. This strand separation can be accomplished by any suitable method including physical, chemical or enzymatic means. One physical method of separating the strands of the nucleic acid involves heating the nucleic acid until it is completely (>99%) denatured. Typical heat denaturation may involve temperatures ranging from about 80° to 105° C. for times ranging from about 1 to 10 minutes. Strand separation may also be induced by an enzyme from the class of enzymes known as helicases or the enzyme RecA, which has helicase activity and in the presence of riboATP is known to denature DNA. The reaction conditions suitable for separating the strands of nucleic acids with helicases are described by Cold Spring Harbor Symposia on Quantitative Biology, Vol. XLIII “DNA: Replication and Recombination” (New York: Cold Spring Harbor Laboratory, 1978), B. Kuhn et al., “DNA Helicases”, pp. 63-67, and techniques for using RecA are reviewed in C. Radding, Ann. Rev. Genetics, 16: 405-37 (1982).

If the original nucleic acid containing the sequence to be amplified is single stranded, its complement is synthesized by adding one or two oligonucleotide primers thereto. If an appropriate single primer is added, a primer extension product is synthesized in the presence of the primer, an inducer or catalyst of the synthesis and the four nucleotides described below. The product will be partially complementary to the single-stranded nucleic acid and will hybridize with the nucleic acid strand to form a duplex of unequal length strands that may then be separated into single strands as described above to produce two single separated complementary strands. Alternatively, two appropriate primers may be added to the single-stranded nucleic acid template and the reaction carried out.

If the original nucleic acid template constitutes the sequence to be amplified, the primer extension product(s) produced will be completely complementary to the strands of the original nucleic acid and will hybridize therewith to form a duplex of equal length strands to be separated into single-stranded molecules.

When the complementary strands of the nucleic acid or acids are separated, whether the nucleic acid was originally double or single stranded, the strands are ready to be used as a template for the synthesis of additional nucleic acid strands. This synthesis can be performed using any suitable method. Generally it occurs in a buffered aqueous solution, preferably at a pH of 7-9, most preferably about 8. Preferably, a molar excess (for cloned nucleic acid, usually about 1000:1 primer:template, and for genomic nucleic acid, usually about 10^{6:}1 primer:template) of the two oligonucleotide primers is added to the buffer containing the separated template strands. It is understood, however, that the amount of complementary strand may not be known if the process herein is used for diagnostic applications, so that the amount of primer relative to the amount of complementary strand cannot be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleoside triphosphates DATP, dCTP, dGTP and TTP are also added to the synthesis mixture in adequate amounts and the resulting solution is heated to about 90°-100°. C for from about 1 to 10 minutes, preferably from 1 to 4 minutes. After this heating period the solution is allowed to cool to room temperature, which is preferable for the primer hybridization. To the cooled mixture is added an appropriate agent for inducing or catalyzing the primer extension reaction, and the reaction is allowed to occur under conditions known in the art. This synthesis reaction may occur at from room temperature up to a temperature above which the inducing agent no longer functions efficiently. Thus, for example, if DNA polymerase is used as inducing agent, the temperature is generally no greater than about 40° C. Most conveniently the reaction occurs at room temperature.

The inducing agent may be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, reverse transcriptase, and other enzymes, including heat-stable enzymes, which will facilitate combination of the nucleotides in the proper manner to form the primer extension products which are complementary to each nucleic acid strand. Generally, the synthesis will be initiated at the 3′ end of each primer and proceed in the 5′ direction along the template strand, until synthesis terminates, producing molecules of different lengths. There may be inducing agents, however, which initiate synthesis at the 5′ end and proceed in the other direction, using the same process as described above.

The newly synthesized strand and its complementary nucleic acid strand form a double-stranded molecule which is used in the succeeding steps of the process. In the next step, the strands of the double-stranded molecule are separated using any of the procedures described above to provide single-stranded molecules.

New nucleic acid is synthesized on the single-stranded molecules. Additional polymerase, nucleotides and primers may be added if necessary for the reaction to proceed under the conditions prescribed above. Again, the synthesis will be initiated at one end of the oligonucleotide primers and will proceed along the single strands of the template to produce additional nucleic acid. After this step, half of the extension product will consist of the specific nucleic acid sequence bounded by the two primers.

The steps of strand separation and extension product synthesis can be repeated as often as needed to produce the desired quantity of the specific nucleic acid sequence. As will be described in further detail below, the amount of the specific nucleic acid sequence produced will accumulate in an exponential fashion.

When it is desired to produce more than one specific nucleic acid sequence from the first nucleic acid or mixture of nucleic acids, the appropriate number of different oligonucleotide primers are utilized. For example, if two different specific nucleic acid sequences are to be produced, four primers are utilized. Two of the primers are specific for one of the specific nucleic acid sequences and the other two primers are specific for the second specific nucleic acid sequence. In this manner, each of the two different specific sequences can be produced exponentially by the present process.

The steps of this process can be repeated indefinitely, being limited only by the amount of primers, polymerase and nucleotides present. The amount of original nucleic acid remains constant in the entire process, because it is not replicated. The amount of the long products increases linearly because they are produced only from the original nucleic acid. The amount of the specific sequence increases exponentially. Thus, the specific sequence will become the predominant species.

Ligase Chain Reaction

In a still further embodiment of the invention, the synthetic nucleic acid may be synthesized from a plasmid nucleic acid of the invention using ligation chain reaction (LCR). LCR is described in PCT Patent Publication No. WO 89/09835, which is incorporated herein by reference. The process involves the use of ligase to join oligonucleotide segments that anneal to the target nucleic acid. LCR results in amplification of an original target molecule and can provide millions of copies of product DNA. Consequently, the LCR results in a net increase in double-stranded DNA.

Typically, the LCR amplification process is initiated on a solution of nucleic acid, preferably DNA, at a concentration of about 1-100 [symbol] g/ml, in a ligation buffer. The ligation buffer is an aqueous solution at a pH of between about 7 and 9, at which the DNA ligase to e used is active, maintained by any standard buffer which the ligase can tolerate (preferably Tris-HCl at a concentration of 5 mM to 50 mM); a small amount of EDTA typically at 0.1-10 [symbol] M; Mn²⁺ or, preferably, Mg²⁺ required for DNA ligase activity, preferably as the chloride salt at 0.2-20 mM concentration; any co-factor required for ligase activity (DPN (otherwise referred to in the art as NAD+) in the case of the E. coli ligase and ATP in the case of the T4 ligase) at a concentration of between 1-100 [symbol] M; a reducing agent such as dithiothreitol or dithioerythritol at about 0.1-10 mM if (as in the case of the T4 ligase) necessary for suitable activity of the ligase being employed; and the segments to be ligated in the amplification process at a very large molar excess, typically 10⁸ to 10¹², relative to the anticipated concentration of target segment present in the solution before initiation of the amplification process. A concentration between about 1 nM and 1 [symbol] M for each of these segments will usually assure that a sufficient molar excess, relative to the concentration of target segment, is present.

Alternatively, the final stem in preparation of ligation buffer can correspond to the initiation of the amplification process. In this alternative, the DNA segments to be ligated are dissolved in a first buffer, which has the same composition as the ligation buffer except that is lacks the DNA sample to be subjected to amplification, and the DNA sample to be subjected to amplification is dissolved in a second buffer, which has the same composition as the ligation buffer except that is lacks the segments to be ligated. The n the ligation buffer is made by combining the first buffer solution, immediately after treatment, if necessary, by heating or another process to render the segments to be ligated single stranded, with the second buffer, immediately after it also has been treated, if necessary, to render single stranded any DNA with the target segment.

Once the necessary DNAs are in single stranded form in the ligation buffer, the first annealing step of the amplification process is carried out. This is accomplished by simply cooling the solution buffer to a temperature near or somewhat below the melting temperature for the duplexes to be formed between the segments to be ligated and the subsegments of the target segment (or complement thereof) to which those segments must hybridize stably for ligation to be catalyzed. It is preferred that this temperature also be in the range of temperatures at which the ligase to be employed retains significant activity.

Once the solution reaches a suitable temperature of catalysis of ligation by the DNA ligase, an aliquot of ligase solution, of preferably significantly smaller volume than (i.e., between about 0.1 and 0.001 times) that of the ligation buffer in which the initial annealing is carried out, is added to the ligation buffer, and thereby, the ligation initiated. The ideal duration of a ligation reaction can be estimated readily by the those skilled in the art, and will depend on several factors, including the particular ligase employed, the concentration of the ligase, the activity of the ligase at the herein described buffer conditions, and the concentrations of target segments and segments to be ligated in the ligation reaction.

After the period for the ligation reaction (1-30 min.), the reaction is terminated by inactivating the ligase, preferably by raising the temperature of the solution to a temperature at which the ligase is essentially inactive. In the case of the E. colt DNA ligase, complete inactivation may be achieved by a few seconds at above 75° C.

After the ligation reaction, the DNA of the solution is strand-separated under high temperature (80-100° C.). Then, as often as necessary (i.e., to amplify a target segment of complement thereof to a concentration that is detectable (i.e., measurable above background, established by suitable controls)) or desirable, reannealing can be carried out as described above for the annealing stem, ligation can be carried out after the reannealing by adding an other aliquot of DNA ligase solution and incubating the solution as described above for the ligation stem, a strand-separation can be carried out as described above after the ligation, and the annealing-ligation-strand-separation cycle can be started again.

Nucleic Acid Sequence Based Amplification

In yet another embodiment of the present invention, synthetic nucleic acid can be synthesized from plasmid template nucleic acid using a nucleic acid based sequence amplification strategy (NASBA). This method is a promoter-directed, enzymatic process that induces in vitro continuous, homogeneous and isothermal amplification of a specific nucleic acid to provide RNA copies of the nucleic acid (U.S. Pat. Nos. 5,130,238, incorporated herein by reference).

The amplification involves the alternate synthesis of DNA and RNA. In this process, single-stranded antisense (−) RNA is converted to single-stranded DNA which in turn is converted to double stranded DNA and becomes a functional template for the synthesis of a plurality of copies of the original single-stranded RNA. A first primer and a second primer are used in the amplification process. A sequence of the first primer or the second primer is sufficiently complementary to a sequence of the specific nucleic acid sequence and a sequence of the first or the second primer is sufficiently homologous to a sequence of the specific nucleic acid sequence. In some instances, both the first primer and second primer are sufficiently complementary and sufficiently homologous to a sequence of the specific nucleic acid sequence, for example, if the specific nucleic acid sequence is double stranded DNA.

The (−) RNA is converted to single-stranded DNA by hybridizing an oligonucleotide primer (the first primer) to 3′ end of the RNA (the first template) and synthesizing a complementary strand of DNA from the first primer (the first DNA sequence) by using a RNA-directed DNA polymerase. The resulting single-stranded DNA (the second template) is separated from the first template by, for example, hydrolysis of the first template by using a ribonuclease which is specific for RNA-DNA hybrids (for example, ribonuclease H). The second template is converted to a form which is capable of RNA synthesis by hybridizing a synthetic oligonucleotide (the second primer), which contains at its 3′ end a sequence which is sufficiently complementary to the 3′ end of the second template and toward its 5′ end a sequence containing a complementary strand of a promoter and antisense sequence of a transcription initiation site, and by synthesizing a second DNA sequence covalently attached to the 3′ end of the second primer using the second template as a template and synthesizing a third DNA sequence covalently attached to the 3′ end of the second template using the second primer as a template, using DNA-directed DNA polymerase. The resulting functional derivative of the second template, which is a third template, is used for the synthesis of a plurality of copies of RNA, the first template, by using a RNA polymerase which is specific for the promoter and transcription initiation site defined by the second primer. Each newly synthesized first template can be converted to further copies of the second template and the third template by repeating the cycle. In addition, repetition of the cycle does not require participation or manipulation by the user.

In one embodiment of this technique, a single-stranded DNA or RNA template could be obtained from a double-stranded DNA (plasmid template), double-stranded RNA or a DNA-RNA hybrid by using chemical, thermal, or possibly enzymatic methods. Then, by using one of the alternative schemes proposed above, the resulting single-stranded DNA or RNA could then be used to generate a template nucleic acid which could function as a first, second or third template. In addition, an alternative scheme involving the first primer and one strand of nucleic acid, and another alternative scheme involving the second primer and the other (complementary) strand of the nucleic acid may be used concurrently to generate template nucleic acids.

Strand Displacement Amplification

Strand displacement amplification refers to an amplification and detection method which operates at a single temperature and makes use of a polymerase in conjunction with an endonuclease that will nick the polymerized strand such that the polymerase will displace the strand without digestion while generating a newly polymerized strand.

Plasmid template nucleic acid is first isolated by the methods described above. Once the nucleic acids are isolated, it will be assumed for purposes of illustration only that the nucleic acid is DNA and is double stranded. In such instances, it is preferred to cleave the nucleic acids in the sample into fragments of between approximately 50-500 bp. This may be done by a restriction enzyme such as HhaI, FokI or DpnI. The selection of the enzyme and the length of the sequence should be such so that the target sequence sought (nucleic acid sequence to be amplified to generate a synthetic product) will be contained in its entirety within the fragment generated or at least a sufficient portion of the target sequence will be present in the fragment to provide sufficient binding of the primer sequence. Other methods for generating fragments include PCR and sonication.

The primers used in this method generally have a length of 25-100 nucleotides. Primers of approximately 35 nucleotides are preferred. This sequence should be substantially homologous to a sequence on the target such that under high stringency conditions binding will occur. The primer also should contain a sequence (toward the 5′ end) that will be recognized by the nicking endonuclease to be used in later steps. The recognition sequences generally, although not necessarily, are non-palindromic. The sequence selected also may be such that the restriction enzyme used to cleave the fragments in the previous step is the same as the nicking endonuclease to be used in later steps.

Once target nucleic acid fragments are generated, they are denatured to render them single stranded so as to permit binding of the primers to the target strands. Raising the temperature of the reaction to approximately 95° C. is a preferred method for denaturing the nucleic acids. Other methods include raising pH; however, this will require lowering the pH in order to allow the primers to bind to the target.

Either before or after the nucleic acids are denatured, a mixture comprising an excess of all four deoxynucleosidetriphosphates, wherein at least one of which is substituted, a polymerase and an endonuclease are added. (If high temperature is used to denature the nucleic acids, unless thermophilic enzymes are used, it is preferable to add the enzymes after denaturation.) The substituted deoxynucleosidetriphosphate should be modified such that it will inhibit cleavage in the strand containing the substituted deoxynucleotides but will not inhibit cleavage on the other strand. Examples of such substituted deoxynucleosidetriphosphates include 2′deoxyadenosine 5′-O-(1-thiotriphosphate), 5-methyldeoxycytidine 5′-triphosphate, 2′-deoxyuridine 5′-triphosphate and 7-deaza-2′-deoxyguanosine 5′-triphosphate.

The mixture comprising the reaction components for target generation and strand displacement amplification can optionally include NMP (1-methyl 2 pyrrolidinone), glycerol, polyp(ethylene glycol), dimethyl sulfoxide and/or formamide. The inclusion of such organic solvents is believed to help alleviate background hybridization reactions.

It should be appreciated that the substitution of the deoxynucleotides may be accomplished after incorporation into a strand. For example, a methylase, such as M Taq I, could be used to add methyl groups to the synthesized strand. The methyl groups when added to the nucleotides are thus substituted and will function in similar manner to the thio substituted nucleotides.

It further should be appreciated that if all the nucleotides are substituted, then the polymerase need not lack the 5′ 3′ exonuclease activity. The presence of the substituents throughout the synthesized strand will function to prevent such activity without inactivating the system.

The selection of the endonuclease used in this method should be such that it will cleave a strand at or 3′ (or 5′) to the recognition sequence. The endonuclease further should be selected so as not to cleave the complementary recognition sequence that will be generated in the target strand by the presence of the polymerase, and further should be selected so as to dissociate from the nicked recognition sequence at a reasonable rate. It need not be thermophilic. Endonucleases, including, but not limited to HincII, HindII, AvaI, Fnu4HI, Tth111I, and NciI are preferred.

According to this method, the primer binds to the target and in the presence of polymerase, deoxynucleosidetriphosphates and [symbol] -thio substituted deoxycytosinetriphosphate, the primer is extended the length of the target while the target is extended through the recognition sequence. In the presence of an endonuclease, the primer strand is nicked at the endonuclease recognition site. In the presence of the polymerase lacking 5′ to 3′ exonuclease activity, the 3′ end at the nick is extended, and downstream the primer strand is displaced from the target strand beginning at the nick to create a reaction product and a new strand is synthesized. In summary fashion, the newly synthesized strand too will be nicked by the endonuclease and the polymerase then will displace this strand generating another until either the reaction is stopped or one of the reagents becomes limiting.

Transcription Reactions

In yet another embodiment of the present invention, a synthetic nucleic acid may be synthesized by transcription reactions in which a messenger RNA molecule is synthesized from a DNA template. Transcription reactions are well known in the art (see for example Ausubel et. al. (1995) Short Protocols in Molecular Biology 3^{rd} Ed., John Wiley and Sons). For example a nucleic acid of interest may be cloned, using methods commonly used in the art, into a vector bearing a promotor for an RNA polymerase such as SP6, T7 or T3. The nucleic acid of interest may then be transcribed from the vector, under appropriate conditions, using the RNA polymerase corresponding to the cloned promoter.

In one embodiment of the transcription reaction, the DNA of interest is cloned, as described above, into a plasmid vector containing a promoter for SP6 or T7 RNA polymerase. The plasmid DNA is isolated and purified by CsCl centrifugation as described above. The plasmid DNA (about 10 [symbol] g) is then cleaved with a restriction endonuclease that cuts just downstream (ideally 50 to 200 bp) of the termination codon of the nucleic acid of interest and does not cut within the coding region of the nucleic acid of interest. The DNA is then purified by phenol extraction and ethanol precipitation, and resuspended in 50 [symbol] l Tris-EDTA buffer. The DNA (1 [symbol] g) may then be mixed with the transcription reaction buffer comprising, 5× ribonucleoside triphosphate mix, 10× SP6/T7 polymerase buffer, 30-60 units Rnasin, and 5-20 units of SP6 or T7 RNA polymerase (depending upon the promoter in the plasmid vector). The reaction mix is incubated at 40° C. for 60 minutes. The transcribed RNA is then extracted by phenol/chloroform/ethanol precipitation, and resuspended in up to 10 [symbol] l TE buffer.

Analytical Procedures

The present invention provides a method for the improvement of the analysis of synthetic nucleic acid molecules which are synthesized from a plasmid template nucleic acid by one or more of the amplification and/or synthetic reactions described herein. Accordingly, the analytical procedure, useful in the present invention, may be selected from the group including, but not limited to gel electrophoresis, anion-exchange chromatography (U.S. Pat. No. 5,866,429, herein incorporated by reference), size-exclusion chromatography (U.S. Pat. No. 4,160,728, herein incorporated by reference), pulse-field electrophoresis, sieving gel electrophoresis, capillary electrophoresis, Northern analysis, Southern analysis, or DNA sequencing.

Gel Electrophoresis

In one embodiment of the present invention the analytical procedure is gel electrophoresis. Gel electrophoresis is a technique which is frequently used in the art to separate nucleic acid molecules in a sample based on size. The gel is typically comprised of algal polysaccharide agarose consisting of alternating units of 3,6-anhydro-L-galactose, glycosylated on O-4, and of D-galactose, glycosylated on O-3, both pyranose, however, for separation of smaller nucleic fragments (up to several hundred nucleotides in length), a polyacrylamide gel may be conveniently used. For analysis, the nucleic acid sample is placed into a well formed in the gel, and an electrical field is applied to the gel. The negative charge of the nucleic acid will result in migration of the nucleic acid through the gel matrix towards the positive pole. The chains of substance that form the gel slow the migration of molecules, and do so progressively more as the molecular size increases. For the mobility/length dependence to be typical, all molecules must be linear, so cyclic forms of nucleic acid must be cleaved.

Gel electrophoresis, useful in the present invention comprises three main steps: The first main step is the preparation of the gel. This is accomplished by preparing a solution of acrylamide, methylenebis-acrylamide or other crosslinking reagents in the buffer of choice. Catalyst (commonly N,N,N′,N′,-tetramethylethylenediamine) and initiator (ammonium persulfate) are then added. The solution is quickly transferred to the electrophoresis chamber (a rectangular area defined by glass or plastic plates is most commonly used), where polymerization takes place. The polymerization transfers the solution into a firm gel, typically within 1 hour. For agarose gels, sufficient agarose to achieve the desired gel percentage (generally between 0.5 and 1.5%) is mixed with electrophoresis buffer (generally either TAE or TBE), and heated to dissolve the agarose. The solution is then cooled to about 55° C., poured into a sealed gel casting platform, and the slot-forming gel comb is set in place at one end of the gel.

The second stem consists of placing the chamber containing polymerized gel (either agarose or polyacrylamide) in the electrophoresis cell where opposite ends of the chamber make immersion contact with two separate buffer reservoirs. In continuous electrophoresis, buffer solution of ionic strength, composition and pH identical to that incorporated into the gel during polymerization is added to each reservoir. In discontinuous electrophoresis a different buffer solution, but generally having a counter ion common with the buffer polymerized in the gel, is added to one of the reservoirs. Electrodes in each reservoir are connected to a direct current power supply. At this point a complete electric circuit exists and the apparatus is ready for application of a sample to be separated. For polyacrylamide gel electrophoresis, some operators, prior to applying the sample, apply potential to the circuit by means of the power supply. This is done to cause migration of residual ammonium persulfate and other charged residues of the gel formation process away from the sample application region of the gel. (J. Petropakis, A. F. Anglemier, and M. W. Montgomery, Anal. Biochem. 46, 594 (1972). This operation is termed “pre-electrophoresis”.

The third main step is the application of sample, establishment of appropriate voltage and current by means of the direct current power supply for a sufficient time to complete the resolution of components in the sample, and the identification, quantification, or isolation of the resolved zones.

A particular variation of gel electrophoresis, useful in the resolution of large nucleic acid molecules, is the technique of pulsed-field gel electrophoresis, in which the molecules are forced to change their direction of migration by periodic changes in the direction of the applied field. For example, the field is typically applied at a 45° angle to the direction of migration, and a subsequent pulse is applied at a equal but opposite angle. By adjusting the multiple variants, i.e., pulse length, strength, angle, etc., large nucleic acid molecules may be analyzed.

A further variation of the gel electrophoresis technique is sieving agarose gel electrophoresis. This technique is particularly useful for the resolution of nucleic acid fragments less than 1 kb. Briefly, sieving agarose gel electrophoresis is performed similar to traditional agarose gel electrophoresis with the exception that a high concentration (3-5%) of a low gelling/melting temperature sieving agarose is used.

Southern Analysis

In one alternate embodiment, an analytical procedure useful in the present invention may be the analysis of DNA molecules by Southern Analysis. DNA synthetic products produced by amplification reactions as described above may be examined using DNA-specific probes which selectively hybridize to predetermined nucleic acid sequences. A nucleic acid sample comprising synthetic DNA may be separated by agarose gel electrophoresis as described herein. Briefly, amplified DNA is fractionated on 1.6% agarose gels, denatured, neutralized, and rapidly downward transferred to Nytran-Plus membrane (Schleicher & Schuell, Keene, N.H.). The membranes are prehybridized in 1.5×SSPE (1×SSPE is 150 mM NaCl, 10 mM monobasic sodium phosphate, pH 7.4, and 1.0 mM EDTA) containing 10% polyethylene glycol, 7% SDS and 200 μg/ml sheared salmon sperm DNA, and subsequently hybridized at 65° C. for 48 hr with a synthetic antisense internal oligonucleotide probe, end-labeled with [ [symbol] -³²P]ATP using T4 polynucleotide kinase (Promega, Madison, Wis.). The blots are then washed and apposed to either x-ray film with an intensifying screen or REFLECTION autoradiography film and screen (DuPont NEN, Wilmington, Del.).

Northern Analysis

In a further alternate embodiment, an analytical procedure useful in the present invention may be the analysis of RNA molecules by Northern Analysis. Ribonucleic acid synthetic products produced by transcription reactions as described above may be examined using RNA-specific probes which selectively hybridize to predetermined nucleic acid sequences. A nucleic acid sample comprising synthetic RNA may be separated by agarose gel electrophoresis as described herein. Briefly, RNA is fractionated on 1.5% agarose gels containing, and downward transferred to a Nytran-Plus membrane using 20×SSC. The blots are then prehybridized with 50% formamide, 5×SSC, 1×PE (1×PE is 50 mM Tris HCl, pH 7.5, 0.1% sodium pyrophosphate, 1.0% SDS, 0.2% polyvinylpyrrolidone, 0.2% Ficoll-400, and 5 mM EDTA) and 200 μg/ml sheared salmon sperm DNA at 65° C., and hybridized with 106 cpm/ml [32P]UTP-labeled antisense riboprobe for 26 hr at 65° C. The blots are then washed and apposed to REFLECTION autoradiography film with a REFLECTION intensifying screen for 9-120 hr at −85° C.

Capillary Gel Electrophoresis

In a preferred embodiment of the present invention, a nucleic acid sample is analyzed by capillary gel electrophoresis. In a further embodiment, the nucleic acid sample is the product of a sequencing reaction carried out prior to analysis by capillary gel electrophoresis. Capillary electrophoresis has been applied widely over traditional gel electrophoresis as an analytical technique because of several technical advantages: (i) capillaries have high surface-to-volume ratios which permit more efficient heat dissipation which, in turn, permit high electric fields to be used for more rapid separations; (ii) the technique requires minimal sample volumes; (iii) superior resolution of most analytes is attainable; and (iv) the technique is amenable to automation, e.g. Camilleri, editor, Capillary Electrophoresis: Theory and Practice (CRC Press, Boca Raton, 1993); and Grossman et al, editors, Capillary Electrophoresis (Academic Press, San Diego, 1992). Because of these advantages, there has been great interest in applying capillary electrophoresis to the separation of biomolecules, particularly in nucleic acid analysis. The need for rapid and accurate separation of nucleic acids, particularly deoxyribonucleic acid (DNA) arises in the analysis of polymerase chain reaction (PCR) products and DNA sequencing fragment analysis, e.g. Williams, Methods 4:227-232 (19920; Drossman et at, Anal. Chem., 62: 900-903 (1990); Huang et at, Anal. Chem., 64:2149-2154 (1992); and Swerdlow et at, Nucleic Acids Research, 18:1415-1419 (1990).

In CE, the physical characteristics of the capillaries are important factors in resolving the components of interest in a sample. The capillaries employed in CE are typically <100 [symbol] m internal diameter (i.d.) and 20-100 cm in length, although the capillaries suitable for use in the present invention are not necessarily limited to these dimensions.

The capillary of the present invention comprises a lumen having a lumenal surface, an inlet, and an outlet. The capillary may be a fused silica capillary, or it may be a channel of appropriate dimensions formed from any suitable material, such as silica, plastic, or glass. The lumen is a bore or a channel through the capillary in which the sample, e.g. amplified nucleic acid, can pass in order to be resolved. In general, any capillary, or capillary-like channel or trough in any microfabricated device is suitable for use in the present invention. Components in the lumen such as matrices, buffers and ampholines allow the sample to be resolved upon application of an electric field.

Capillaries used in CE may be comprised of fused silica, which is known to impart a net negative charge to the inner surface of the capillary. The inner surface of the capillaries may in this case be coated with polymers or other compositions which result in a surface with the desired charge characteristics, e.g. charge-neutrality. Capillaries formed from other materials besides fused silica, such as plastic, may also be used. This may alleviate the necessity coating of the lumenal surface to achieve the desired charge characteristics. In addition, an external polymeric coating is used which produces a surprisingly flexible narrow-bore capillary that would otherwise be extremely fragile.

Separation of the amplified nucleic acid requires the presence within the lumen of the capillary of an appropriate buffer containing a polymeric network. The buffer provides an environment that is chemically compatible for the separation of nucleic acid, and also acts as the solvent for the polymeric matrix. The combination of the charge neutral lumenal surface and the buffer containing the polymeric network may provide for the separation of molecules by two different mechanisms. While not bound by theory, a coating of the wall to provide charge neutrality, may provide insulation of the analyte from the charged surface, and may provide a viscous layer to reduce electroosmotic flow (EOF) at the lumenal surface-solution interface. The reduction in EOF may allow the separation of nucleic acid by virtue of the differences in their charge-to-mass ratio. The polymeric network may also provide a sieving medium, by which molecules having similar charge-to-mass ratios may be separated by their mass-equivalent hydrodynamic volume (i.e., size). Either mechanism individually, or the combination of the two mechanisms, may effect the resolution of amplified nucleic acid of the invention.

The polymeric network can either be a network polymerized within the capillary or a free-flowing network. A free flowing network, is pump-able into and out of the capillary, as opposed to a gel or matrix that is fixed within the capillary and suitable for single use. Polymerized linear matrices such as linear polyacrylamide may be used as a polymeric matrix, or as a coating. Capillaries coated with linear polyacrylamide or containing cross-linked acrylamide are presently commercially available.

As used herein, the term “separation medium” refers to the medium in a capillary in which the separation of analyte components takes place. Separation media typically comprise several components, at least one of which is a charge-carrying component, or electrolyte. The charge-carrying component is usually part of a buffer system for maintaining the separation medium at a constant pH. Media for separating polynucleotides, or other biomolecules having different sizes but identical charge-frictional drag ratios in free solution, further include a sieving component. In addition to such conventional components, the separation medium of the invention comprise a surface interaction component. In the case of polynucleotide separations, the sieving component may be the same or different than the surface interaction component, but is usually different. The surface interaction component comprises one or more uncharged water-soluble silica-adsorbing polymers having the physical properties set forth above. Preferably, such one or more uncharged water-soluble silica-adsorbing polymers are non-hydroxylic. In further preference for polynucleotide separations, the sieving component, herein referred to as the “polymeric network” of the separation medium of the invention comprises one or more uncrosslinked, particularly linear, polymers. Preferably, the components of the separation medium of the invention are selected so that its viscosity is low enough to permit rapid re-filling of capillaries between separation runs. In the presence of a polymeric network component, viscosity is preferably less than 5000 centipoise, and more preferably, less than 1000 centipoise.

A variety of free-flowing polymeric networks may be used. Free-flowing matrices may be comprised of cellulosic material. Other free-flowing matrices, such as polyethylene oxide (PEO), polyethylene glycol (PEG), and the linear acrylamides, also may be used. Specifically, cellulosic matrices such as hydroxypropylmethyl cellulose (HPMC), hydroxyethyl cellulose (HEC) or methyl cellulose may be used at varying concentrations.

The polymeric network is typically suspended in a biological buffer. Selection of a buffering system is a crucial step in devising a separation scheme. The buffering system must maintain the pH compatible for the separation of nucleic acid. Furthermore, at similar pH's a particular buffering system results in successful separation, while others may not. Thus, selection of a buffering system is a key step for successful resolution of the components of interest.

The sample may be introduced into the inlet of the capillary by various techniques. The most commonly used techniques are electrokinetic injection or hydrodynamic injection. In electrokinetic injection, a low voltage (typically at about 6 kV for about 20 sec-1 min.) is used initially to allow the sample to enter into the capillary, whereas in hydrodynamic injection, pressure or suction is used to drive the sample into the capillary.

Additional parameters for electrophoresis include maintaining the capillary temperature at about 20-40° C. This can only be accomplished in capillary electrophoresis when large electric fields are applied because the capillary provides a high surface-to-volume ratio which allows for very efficient dissipation of Joule heat.

One difficulty encountered with capillary electrophoresis, is that the polymeric network is highly sensitive to the amount of DNA loaded into the capillary. Moreover, the large size of the plasmid template nucleic acid in the nucleic acid samples described herein, may clog the pores of the polymeric network, thereby impairing the analysis of the sample. Therefore, according to the present invention, the nucleic acid sample is treated, following amplification, but prior to analysis, with a substance which cleaves the template nucleic acid without substantially cleaving the synthetic nucleic acid.

DNA Sequencing

In a further embodiment of the present invention, a nucleic acid sample may be analyzed by sequencing, wherein the nucleotide sequence of a synthetic nucleic acid is elucidated. To determine the sequence of a nucleic acid, the nucleic acid sample is first subjected to a sequencing reaction, such as Sanger dideoxy sequencing as described above (Sanger et. al., (1977) Proc. Natl. Acad. Sci. USA 74: 5463, which is incorporated herein by reference). This method relies upon the template-directed incorporation of nucleotides onto an annealed primer by a DNA polymerase from a mixture containing deoxy- and dideoxynucleotides. The incorporation of dideoxynucleotides results in chain termination, the inability of the enzyme to catalyze further extension of that strand. Subsequent electrophoretic separation of reaction products results in a “ladder” of extension products wherein each extension product ends in a particular dideoxynucleotide complementary to the nucleotide opposite it in the template. Extension products may be detected in several ways, including for example, the inclusion of isotopically-or fluorescently-labeled primers, deoxynucleotide triphosphates or dideoxynucleotide triphosphates in the reaction.

The sequencing reaction product may then be analyzed by a variety of means, most generally by capillary gel electrophoresis, and/or polyacrylamide gel electrophoresis. Both of these techniques have been described in detail above, and are further described in numerous texts and laboratory manuals, including Short Protocols in Molecular Biology (Ausubel et. al. (1995) 3^{rd} Ed. John Wiley & Sons, Inc.).

Cleavage of Template Nucleic Acid

The present invention relates to a method of improving the analysis of a nucleic acid sample comprising a template nucleic acid and a synthetic nucleic acid derived from the template by a synthetic reaction of the invention, comprising treating the nucleic acid sample with a substance which cleaves the template without substantially cleaving the synthetic nucleic acid.

In a preferred embodiment of the invention, the substance is a restriction enzyme which selectively cleaves the template but not the synthetic nucleic acid. For example, the enzyme may substantially cleave unmodified residues without substantially cleaving modified residues. For example synthetic nucleic acid may be synthesized from a template nucleic acid, wherein the template is comprised of unmodified residues, wherein either methylated adenine, or cytosine residues are included in the synthesis reaction in place of unmethylated adenine or cytosine. Accordingly, the synthetic product will incorporate the modified nucleotides during synthesis. Subsequent to the synthetic reaction, and prior to analysis of the synthetic product, the nucleic acid sample may be treated with a restriction enzyme which selectively cleaves unmodified residues (template) but not modified residues (synthetic nucleic acid). For example, if methylated adenine is incorporated into the synthetic nucleic acid, the nucleic acid sample may be treated with one or more of AlwI, BclI, BsaBI, BspDI, BspEI, BspHI, ClaI, DpnII, HphI, MboI, MboII, NruI, TaqI, or XbaI. If methylated cytosine is incorporated into the synthetic nucleic acid, the nucleic acid sample may be treated with one or more of Acc65I, AlwNI, ApaI, AvaII, BalI, BpmI, BslI, Bsp120I, BssKI, EaeI, EcoO109I, EcoRII, MscI, PflMI, PpuMI, Sau96I, ScrGI, SexAI, SfiI, StuI. Thus, the desired synthetic nucleic acid which is methylated, is not cleaved, while the undesired template nucleic acid, which is unmethylated, is cleaved, thus, according to the present invention, providing improved analysis of the synthetic nucleic acid.

In a preferred embodiment of the invention, the substance is a restriction enzyme which selectively cleaves the template but not the synthetic nucleic acid. For example, the enzyme may selectively cleave modified, or methylated residues without substantially cleaving unmodified, or unmethylated residues. Thus, as described above, a modified plasmid template nucleic acid which is selectively cleaved, useful in the present invention, may be generated in dam⁺ E. coli. Plasmid template nucleic acid synthesized in this manner, contains methylated adenine residues in the sequence GATC, which occurs about every 250 bp. During the sequencing or other amplification reaction the synthetic product is synthesized using unmethylated free nucleotides form the cycle sequencing or other amplification reaction mix. Thus, the desired sequencing or amplified product is unmethylated while the problematic plasmid template DNA is methylated.

As provided by the present invention, the methylated template nucleic acid may be selectively cleaved with a restriction enzyme which is specific for methylated residues. A preferred restriction enzyme of this type is DpnI, which selectively cleaves at GATC sequences only when the adenine residue is methylated, and is commercially available from several scientific vendors. Accordingly, a nucleic acid sample comprising plasmid template nucleic acid derived from dam⁺ E. coli, and synthetic nucleic acid synthesized from the template by an amplification reaction useful in the present invention, may be treated with DpnI, under conditions which facilitate optimal DpnI enzymatic activity, wherein DpnI will selectively cleave the template nucleic acid but not substantially cleave the synthetic nucleic acid. Conditions which provide optimal enzymatic activity for DpnI are known in the art, and, moreover, are given in the literature provided upon purchase of the enzyme from a commercial source (i.e., Life Technologies, Rockville, Md.).

In a preferred embodiment the nucleic acid sample treated with DpnI is the product of a sequencing reaction to be analyzed by capillary gel electrophoresis. One of the difficulties with capillary sequencers is that they are very sensitive to the amount and size of DNA loaded into the capillary. Too much DNA can clog the capillary yielding unusable sequencing data. When using double stranded plasmid DNA as the template for cycle sequencing reactions, the large vector DNA is necessarily present in the sample. The larger vector DNA can increase the viscosity of the sample within the capillary and effectively clog the capillary. Given that the vector DNA is not the portion of the nucleic acid sample that is of interest, the viscosity and overall size of the nucleic acid sample may be reduced by treating the sample with DpnI to selectively cleave the plasmid template. The DpnI recognition site occurs approximately every 250 bases, thus the plasmid template nucleic acid may be, in some instances, be cleaved to produce approximately 250 bp fragments.

The improvement of capillary gel electrophoresis of a nucleic acid sequencing reaction may be determined by an increase in the quantity or quality of data which is obtained from the electrophoretic separation. For example, in capillary based DNA sequencing, an improvement in the sequencing could be the resolution of a higher number of bases from a given sample. In preferred embodiments, capillary based DNA sequencing following treatment of the nucleic acid with a selective cleaving substance useful in the present invention, such as DpnI, would resolve about 10-20% more bases than capillary based sequencing of a nucleic acid sample which has not been treated with a selective cleaving substance, preferably about 20-50%, more preferably about 50-80%, and most preferably about 80-100% more bases.

Alternatively, a nucleic acid sample of the present invention may be cleaved with an enzyme which selectively cleaves double stranded nucleic acid, without substantially cleaving single stranded nucleic acid. Nucleic acid samples of the which are products of a sequencing reaction or a transcription reaction are generally comprised of double stranded plasmid template nucleic acid and single stranded synthetic nucleic acid (RNA in the case of a transcription reaction). Following a sequencing reaction and prior to analysis by capillary gel electrophoresis or other analytical technique a nucleic acid from a sequencing reaction may be treated with an enzyme, under optimal conditions for a given enzyme, which selectively cleaves double stranded nucleic acid, but not single stranded nucleic acid. The enzyme may be selected from the group including, but not limited to Alu I, Bbv I, Dpn I, FnuD II, Fok I, Hpa II, Hph I, Mbo I, Mbo II, Msp I, Sau3A I, and SfaN I (New England Biolabs Catalog, Beverly, Mass.). Conditions which provide optimal enzymatic activity for the above listed enzymes are known in the art, and, moreover, are given in the literature provided upon purchase of the enzyme from a commercial source.

An alternative embodiment of the present invention comprises the use of adapter sequences incorporated into the plasmid template nucleic acid that function to protect the synthetic nucleic acid from cleavage by a restriction enzyme while the template nucleic acid is cleaved. Preferably, the methods of the invention employ a selected adaptor comprising a cleavage site, such as a restriction enzyme recognition site. Modified nucleotides may optionally be added to the amplification reactions, useful in the present invention, so that they are incorporated into the synthetic nucleic acid so as to permit differential cleavage of template and synthetic nucleic acid. The presence or absence of modified nucleotides results in a difference in susceptibility to a selected reagent substantially incapable of cleaving at a modified site, or alternatively, substantially permitting cleavage at a modified site. Preferably, this is accomplished by the selection of a restriction enzyme which, in the presence of a selected modified nucleotide, is either rendered substantially capable or substantially incapable of cleavage at a modified site. In preferred embodiments, the modified nucleotides can be one of many modified nucleotides, for example the particularly preferred methylated nucleotide bases such as 5-methyl-dCTP, as well as other analogs such as 2′-deoxyriboinosine, 5-iso-2′-deoxyribocytosine, or 5-mercuri-2′-deoxyriboguanosine.

An example of a selected adaptor rendering desired nucleic acids resistant to cleavage by a restriction enzyme utilizes the restriction enzyme recognition site for Eam 1104I, which will not cleave DNA when its CTCTTC recognition site is methylated. An adaptor with the CTCTTC sequence may be incorporated into the plasmid template nucleic acid, using molecular cloning techniques known to those of skill in the art (Ausubel et. al. (1995) Short Protocols in Molecular Biology John Wiley and Sons). The adaptor may be positioned in the plasmid such that primers used in an amplification reaction of the invention will anneal to the plasmid in such a way as to incorporate the Eam 1104I recognition sequence into the newly synthesized nucleic acid molecule. Inclusion of a selected modified nucleotide, such as methyl-dCTP, in the amplification reaction, results in a synthetic nucleic acid which is methylated at the Eam 1104I recognition site. The use of the recognition site sequence of Eam 1104I in an adaptor is a particularly preferred embodiment of the invention because the incorporation of a single methylated cytosine residue in the Eam 1104I site will protect a nucleic acid from cleavage. Accordingly, a nucleic acid sample which is produced in this manner may be treated with Eam 1104I, under conditions for optimal enzymatic activity, which will selectively cleave the plasmid template nucleic acid containing unmodified Eam 1104I recognition sites, but will not substantially cleave the synthetic nucleic acid containing modified Eam 1104I sites. Further examples of the use of adaptors in the generation of selectively susceptible nucleic acid populations can be found in U.S. Pat. No. 6,060,245, herein incorporated by reference.

All literature publications, patents and patent applications referred to herein are incorporated herein in their entirety by reference.

### EXAMPLE 1

Capillary Sequencing

A series of tests were performed to determine the effect of Dpn I on the sequencing efficiency for sequencing reactions using purified plasmid DNA as template and run on the MegaBace 1000 Capillary DNA Sequencer (Amersham Pharmacia Biotech, Piscataway, N.J.). Two duplicate sequencing reactions were run using a 96 well plate of purified plasmid DNA from the HUCLR library as template. The 96 clones were screened for an insert and contamination prior to selection. The reactions were run with the following conditions:

5 [symbol] l of unnormalized purified plasmid DNA was added to 4 [symbol] l of Big Dye Terminator Cycle Sequencing Ready Reaction Mix and 1 ul of HUCLR (6.2 pmol/ [symbol] l) vector specific primer. The reactions were cycled on a Perkin Elmer 9600 Thermal Cycler using the following program:

| Temperature | Time | Cycles |
|---|---|---|
| 96° C. | 0:10 | |
| 45° C. | 0:15 | |
| 60° C. | 4:00 | |
| 4° C. | Hold | |

2. One of the reaction plates had 10 [symbol] l of the Dpn I cocktail (see table below) added to each well. The plate was incubated at 37° C. for 2 hours and then denatured at 95° C. for 2 minutes to stop all enzyme activity.

| Reagent | Concentration | Volume/reaction |
|---|---|---|
| Dpn I | 5 U/ [symbol] 1 | 0.2 [symbol] 1 (1 U) |
| Optimal Buffer #7 | 10X | 2 [symbol] 1 (1×) |
| ddH₂O | — | 7.8 [symbol] 1 |

3. Both plates of reactions were purified using G50 Sephadex filter plates. The entire reaction volume was added to center of the filter columns without touching the resin. The samples were spun at 910 xg for 5 minutes and collected in a clean 96 well plate. The samples were dried in a Savant Speedvac for approximately 1 hour.

4. The reactions were resuspended in 5:1 Formamide to ddH₂O and run on the Megabace 1000 Capillary Sequencers under the same conditions.

| | | |
|---|---|---|
| Injection Voltage: | 2 | kv |
| Injection Time: | 1 | minute |
| Run Voltage: | 6 | kv |
| Run Time: | 180 | minutes |

The sequence from both plates was analyzed and the results have been compiled below.

TABLE 1

READ
LENGTH

| REACTION | # of PASSES | 250-500 bases | >500 bases |
|---|---|---|---|
| W/O Dpn I | 3 | 3 | 0 |
| W/Dpn I | 58 | 5 | 53 |

The present results demonstrate that treatment of a nucleic acid sample, generated by a sequencing reaction, with DpnI to selectively cleave the template nucleic acid provides improved sequence resolution over samples not treated with DpnI. Of the samples not treated with DpnI, none of the samples were resolved to more than 500 bases. In contrast, of the samples treated with DpnI, 91% of the samples were resolved to greater than 500 bases. Thus, the results demonstrate an improvement in sequence resolution by capillary gel based sequencing following selective cleavage of the template nucleic acid.

### EXAMPLE 2

Polymerase Chain Reaction

To determine the effect of plasmid template cleavage on the analysis of polymerase chain reaction synthetic products, the following protocol is carried out.

Two duplicate amplification reactions will be carried out to compare methods of selectively cleaving template nucleic acid: one amplified nucleic acid sample will be treated with an enzyme that selectively cleaves modified DNA prior to analysis by agarose gel electrophoresis, and the other will be subjected to agarose gel electrophoresis without pretreatment to cleave the template. Here, the enzyme which selectively cleaves the modified template DNA is DpnI, which selectively cleaves at the consensus sequence GATC only when the cytosine residue is methylated.

Purified plasmid DNA containing the nucleic acid of interest is isolated from dam+E. coli, and thus possess methylated cytosine residues, using the Wizard® Minipreps system from Promega (Madison, Wis.). Amplification of the plasmid template is preformed on a Perkin Elmer 9600 Thermal Cycler in a 13 [symbol] l reaction volume consisting of 12.5 mM Tris-HCl (pH 8.3) containing 62.5 mM KCl, 2.5 mM MgCl₂, 200 [symbol] M deoxynucleotide triphosphates, 0.5 [symbol] M of primers, 0.5 [symbol] l of purified plasmid DNA, and 0.3 units of AmpliTaq Gold DNA polymerase (PE Applied Biosystems, Norwalk, Conn.) with the following cycling parameters: initial denaturation/enzyme activation, 95° C., 10 min.; (35 cycles) denaturation/enzyme activation, 94° C., 45 s; annealing, transcript-specific temperature, 30 s; primer extension, 72° C., 45 s; final extension, 72° C., 5 min. Amplification is conducted using primers designed specifically to anneal to the gene of interest.

Following amplification, to one nucleic acid sample is added 10 [symbol] l of the following DpnI cocktail to selectively cleave the plasmid template nucleic acid without substantially cleaving the synthetic product: 1 unit DpnI; 2 [symbol] l of 10× Optimal Buffer #7; 7.8 [symbol] l sterile distilled H₂O. The sample is incubated with DpnI at 37° C. for 2 hours to achieve maximal template cleavage, and then denatured at 95° C. for 2 minutes to inactivate the enzyme.

The amplified, DpnI treated and un-treated samples are subsequently resolved on 2% agarose-GelTwin II (J. T. Baker, Phillipsburg, N.J.) gels and visualized by ethidium bromide staining under UV illumination. The stained gels are photographed, and scanned, on a flatbed scanner to a computer. The gel images are then imported into NIH Image, or other comparable image analysis software. The area of each nucleic acid gel band is circumscribed by a user, and the software will subsequently calculate the pixel intensity, and pixel density, and create a plot of pixel intensity vs. area. The area under the resulting curve may be calculated and compared between the two samples to determine the efficacy of DpnI treatment.

According to the invention, reduction in the molecular weight of the DNA template by selective cleavage with DpnI is expected to result in higher signal intensity and better resolution of the synthetic nucleic acid. In one embodiment, the synthetic nucleic acid may be used as a probe for Southern analysis. According to the invention, selective cleavage of the template nucleic acid is expected to yield a higher specific activity probe generated from the synthetic nucleic acid and thus higher hybridization sensitivity.

### EXAMPLE 3

Transcription Reactions

In order to improve the analysis of the product of a transcription reaction following selective cleavage of the synthetic nucleic acid, the following protocol may be used. In this example, the transcription reaction includes a DNA template and an RNA product, and the DNA template is selectively cleaved whereas the RNA product is not cleaved.

A DNA template is prepared for use in the transcription reaction as follows. The nucleic acid of interest is cloned into the plasmid pBluescript II KS⁻ by first cleaving both pBluescript and the nucleic acid of interest with a one or more restriction enzymes so as to create complementary ends on each molecule to facilitate ligation of the nucleic acid of interest into pBluescript. The nucleic acid of interest (insert) is mixed with the plasmid vector at a molar ratio of 2:1 (insert:vector). The insert/vector are ligated in the following reaction: prepared vector (amount added based on picomole ends/micrograms of DNA); prepared insert (amount added based on picomole ends/micrograms of DNA); 10 mM rATP (pH 7.0); 10× ligase buffer; 2 units T4 DNA ligase. The reaction is incubated for 2 hours at room temperature (22° C.) or overnight at 4° C. Between 1 and 2 [symbol] l of the ligation mix is then transformed into appropriate competent cells such as dam+E. coli, and plated on appropriate selective media. Positive clones are then selected and incubated overnight to amplify the cell population bearing the cloned insert. The recombinant plasmid may then be purified using the Wizard® Minipreps system from Promega (Madison, Wis.).

The plasmid is cleaved with BssHII to excise the insert with the T4 and T7 promoters of pBluescript intact. The transcription reaction is then performed in the following reaction: 5× Transcription buffer; 1 [symbol] g of BssHII treated DNA template; 10 mM rATP; 10 mM rCTP; 10 mM rGTP; 10 mM rUTP; 0.75 M dithiothreitol; 10 units of T3 or T7 RNA polymerase; sterile distilled H₂O up to 25 [symbol] l. The reaction is incubated at 37° C. for 30 minutes.

The transcription reaction sample is subsequently divided into three test samples: one is treated with Dnase (1 unit of enzyme per 2 [symbol] g of DNA; 37° C. for 30 min.), one is treated with AluI to selectively cleave double stranded nucleic acid, and one sample will serve as a control. The Dnase, AluI treated and un-treated samples are subsequently resolved on 2% agarose-GelTwin II (J. T. Baker, Phillipsburg, N.J.) gels and visualized by ethidium bromide staining under UV illumination. The stained gels are photographed, and scanned on a flatbed scanner to a computer. The gel images are then imported into NIH Image, or other comparable image analysis software. The area of each nucleic acid gel band is circumscribed by a user, and the software will subsequently calculate the pixel intensity, and pixel density, and create a plot of pixel intensity vs. area. The area under the resulting curve may be calculated and compared between the two samples to determine the efficacy of DpnI and AluI treatment.

According to the invention, reduction in the molecular weight of the DNA template by cleavage with Dnase or AluI is expected to result in higher signal intensity and better resolution of the RNA product of the transcription reaction. In one embodiment, the synthetic nucleic acid may be used as a probe for Northern analysis. According to the invention, selective cleavage of the template nucleic acid is expected to yield a higher specific activity riboprobe generated from the synthetic nucleic acid and thus higher hybridization sensitivity.

### OTHER EMBODIMENTS

Other embodiments will be evident to those of skill in the art. It should be understood that the foregoing detailed description is provided for clarity only and is merely exemplary. The spirit and scope of the present invention are not limited to the above examples, but are encompassed by the following claims.

1. A method of preparing a nucleic acid sample for an analytical procedure, said sample comprising template nucleic acid and synthetic nucleic acid, wherein said template and synthetic nucleic acid comprise DNA, said method comprising treating said sample with a substance that cleaves said template nucleic acid without substantially cleaving said synthetic nucleic acid, and subjecting said treated sample to an analytical procedure, wherein said analytical procedure is selected from the group consisting of anion-exchange chromatography, size-exclusion chromatography, pulse-field electrophoresis, polyacrylamide gel electrophoresis, sieving gel electrophoresis, or Northern analysis.

2. a transcription reaction wherein a nucleic acid sample is generated comprising template nucleic acid and synthetic RNA, the improvement whereby after the transcription reaction and immediately prior to the analysis of the RNA sample, said nucleic acid sample is treated with a substance that cleaves the template nucleic acid and does not substantially cleave the RNA, wherein said substance is a restriction enzyme.

3. The method of claim 1, wherein said synthetic nucleic acid is synthesized from said template.

4. The method of claim 1, wherein said synthesized nucleic acid is synthesized in a reaction selected from the group consisting of sequencing reactions, self-sustained sequence replication amplification, transcription based amplification, strand displacement amplification, ligation chain reaction, nucleic acid-based amplification, or oligonucleotide ligation assay.

5. The method of claim 6, wherein the template nucleic acid is DNA and the synthetic nucleic acid is RNA.

6. The method of claim 1, wherein said substance is a restriction enzyme.

7. The method of claim 11, wherein said restriction enzyme specifically cleaves nucleic acid comprising modified residues, without substantially cleaving unmodified residues.

8. The method of claim 11, wherein said restriction enzyme specifically cleaves nucleic acid comprising unmodified residues, without substantially cleaving modified residues.

9. The method of claim 11, wherein said restriction enzyme specifically cleaves double stranded nucleic acid, without substantially cleaving single stranded nucleic acid.

10. The method of claim 1, wherein said template nucleic acid is a double stranded nucleic acid.

11. The method of claim 1, wherein said synthetic nucleic acid is a single stranded nucleic acid.

12. The method of claim 15, wherein said double-stranded template is produced in cells which incorporate methylated adenine residues into DNA molecules during replication.

13. The method of claim 17, wherein said cell is a dam+ E. coli cell.

14. The method of claim 6, wherein said synthetic RNA is synthesized from said template.

15. The method of claim 6, wherein said restriction enzyme specifically cleaves nucleic acid comprising modified residues, without substantially cleaving unmodified residues.

16. The method of claim 6, wherein said restriction enzyme specifically cleaves nucleic acid comprising unmodified residues, without substantially cleaving modified residues.

17. The method of claim 6, wherein said restriction enzyme specifically cleaves double stranded nucleic acid, without substantially cleaving single stranded nucleic acid.

18. The method of claim 6, wherein said template nucleic acid is a double stranded nucleic acid.

19. The method of claim 6, wherein said synthetic nucleic acid is a single stranded nucleic acid.

20. The method of claim 23, wherein said double-stranded template is produced in cells which incorporate methylated adenine residues into DNA molecules during replication.

21. The method of claim 25, wherein said cell is a dam+ E. coli cell.
